(19) United States
(12) Patent Application Publication
(10) Pub. No.: US 2005/0032062 A1
(43) Pub. Date: Feb. 10, 2005

(54) Methods for the assessment and prognosis of sarcoidosis

(76) Inventors: Salah-Dine Chibout, Tagolsheim (FR); Olivier Grenet, Blotzheim (FR); Jeanne Kehren, Bantzenheim (FR); Frank Stadtler, Eimeldingen (DE)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/497,349

(22) PCT Filed: Nov. 28, 2002
(86) PCT No.: PCT/EP02/13448

Related U.S. Application Data
(60) Provisional application No. 60/334,264, filed on Nov. 29, 2001.

(51) Int. Cl. C12Q 1/68; G06F 19/00; G01N 33/48; G01N 33/50

(52) U.S. Cl. 435/6; 435/7.92

(57) ABSTRACT

This invention identifies genes and the mRNA and polypeptide expression products of these genes which can be used as biomarkers to provide diagnostic and prognostic information in patients with sarcoidosis. These biomarkers can also be used to monitor the severity and progression of sarcoidosis and to identify drugs useful in treating the disease.

### BACKGROUND OF THE INVENTION

This invention relates to methods for the monitoring, prognosis and treatment of sarcoidosis. In particular, the invention relates to the use of gene expression analysis or the measurement of gene expression products to determine the type, prognosis and need for treatment of sarcoidosis.

### DESCRIPTION OF THE RELATED ART

Sarcoidosis is a disease of unknown cause. It is characterized by the presence of non-caseating granulomas in one or more organ systems. The most common sites of involvement are the lungs and the lymph nodes in the mediastinum and hilar regions. However, sarcoidosis is a systemic disease and a variety of organ systems or tissues may be the source of primary or concomitant clinical manifestations and morbidity. The clinical course of sarcoidosis is extremely variable, and ranges from a mild or even asymptomatic disease with spontaneous resolution to a chronic progressive disease leading to organ system failure and, in 1-5% of cases, death. See Cecil Textbook of Medicine, 21^{st} Edition, Goldman L., Bennett, J. C. Editors, W. B. Saunders Company, Philadelphia, 2000 (esp. pages 433-436).

Sarcoidosis is relatively common in northern Europe, North America and Japan. In the United States, sarcoidosis is more frequent In blacks than in whites, with age-adjusted annual incidences reported as 35.5 and 10 per 100,000 respectively. The age of peak incidence of sarcoidosis is in the 20's and 30's, with women affected slightly more often then men.

The cause of sarcoidosis is unknown. However, a substantial body of information suggests that immune mechanisms are important in disease pathogenesis and a variety of exogenous agents, both infectious and non-infectious, have been hypothesized as possible cause of sarcoidosis. These agents include mycobaceria, fungi, spirochetes, and the agent associated with Whipple's disease.

The pathogenesis of sarcoidosis involves the triggering of an oligoclonal expansion of CD4 lymphocytes of the TH¹ phenotype with the production of IL-2 and IFN-γ. IL-2 causes the proliferation of more CD4 cells, which elaborate cytokines that recruit macrophages into the forming granuloma.

Sarcoidosis is especially notable for its protean clinical manifestations and variable and unpredictable course. The disease can affect any organ system and the clinical presentation and natural history of the disease, even in a given organ system, are highly variable. The respiratory system is most commonly affected and almost 90% of patients demonstrate intrathoracic involvement on a chest radiograph. Patients may or may not develop extrathoracic disease, which may be subclinical or, alternatively, may be the predominant component of the clinical picture. The organ systems which are commonly affected, other than the lungs, include the skin, eye, heart, liver, and central nervous system.

The diagnostic evaluation of sarcoidosis presents several problems to the clinician. The diagnosis can be confirmed by the finding of well-formed non-caseating granulomas in one or more affected organ systems or tissues, with the appropriate additional studies to exclude other causes of granulomas, primarily infectious diseases. The diagnosis is often made initially by way of a chest radiograph. In addition, computed tomography (CT) or magnetic resonance imaging (MRI) may be helpful. Tissue biopsy of lung or other tissue may show the typical non-caseating granulomas consisting of epitheloid histiocytes surrounded by a rim of variable numbers of lymphocytes.

The chest radiograph is used to determine the staging of intrathoracic sarcoidosis. A commonly used method is to assign a stage of 0-4 according to the presence or absence of hilar adenopathy and parenchymal disease, e.g., pulmonary infiltration or fibrosis. Thus there are five roentgenographic stages or types of intrathoracic changes (Table 1 below). See Silzbach, L. E. (1967) Med. Clin. N. Am. 51:483; Hunninghake, G. W. (1999) Sarcoidosis, Vasculitis and Diffuse Lung Diseases; 16:149-173 and Cecil Textbook of Medicine, 21^{st} Edition, Goldman L., Bennett, J. C. Editors, W. B. Saunders Company, Philadelphia, 2000 (Pages 433-436).

TABLE 1
Chest Radiographic Staging of Sarcoidosis*

Stage 0 describes no visible intrathoracic findings.
Stage 1 is bilateral hilar lymphadenopathy (BHL), which may be
accompanied by paratracheal adenopathy. Although lung fields are clear of
infiltrates, parenchymal granulomas are often found in lung tissue
biopsies.
Stage 2 is bilateral hilar adenopathy (BHL) accompanied by parenchymal
infiltration.
Stage 3 is parenchymal infiltration without bilateral hilar adenopathy
(BHL)
Stage 4 consists of advanced pulmonary fibrosis with evidence of honey-
combing, hilar retraction, bullae, cysts, and emphysema.
*Classification is based on the posteroanterior chest radiogram only.

However, there is no simple test that can predict the natural history of sarcoidosis, which, as discussed, is highly variable. The disease may spontaneously resolve, or may be persistent and chronic and require extensive treatment with steroids or other powerful and toxic immunosuppresive drugs. The enormous clinical variability of the disease makes it very difficult to decide whether and when therapy should be instituted. Two-thirds of patients with sarcoidosis will have spontaneous remissions, but up to 20% will have a chronic functional deficit, and the disease is fatal in 1-5%. In some patients, the clinical presentation and radiographic staging allow some determination of prognosis. However, the lack of sensitive prediction and disease activity markers, combined with the high rate of spontaneous remission, make it difficult to know when to treat and for how long, as well as the likely outcome in a significant number of patients.

Considerable attention has been directed to the identification of environmental and genetic factors leading to the development of sarcoidosis. An antigenic cause appears likely, insofar that granuloma formation appears to be a generic response of the body to resistant intra-cellular antigens, e.g., mycobacteria or beryllium. In addition, the density of HLA class II molecules on lung effector cells has been shown to be increased, See Spurzem, J. R., et al. 1989 Expression of HLA class II genes in alveolar macrophages of patients with sarcoidosis. Am. Rev. Respir. Dis. 140:89-94; Haslem, P. L. et al. 1990 Increases in HLA-DQ, DP, DR and transferrin receptors on alveolar macrophages in sarcoidosis and allergic alveolitis compared with fibrosing alveolitis Chest 97:651-661, and T-cell receptors (TCR) show evidence of recent antigenic stimulation, See Du Bois, R. M. et al. 1992 T-lymphocytes that accumulate in the lung in sarcoidosis have evidence of recent stimulation of the T-cell antigen receptor. Am. Rev. Respir. Dis. 145:1205-1211.

Numerous infectious and non-infectous agents have been proposed as causal in sarcoidosis but none have been consistently isolated from patients with the disease. See Saboor, S. A., et al. 1992 Detection of mycobacterial DNA in sarcoidosis and tuberculosis with polymerase chain reaction Lancet 339:1012-1015; Almenoff, P. L. et al. 1996 Growth of acid-fast L forms from the blood of patients with sarcoidosis, Thorax 51:530-533; Di Alberti, L., et al. 1997 Human herpesvirus 8 variants in sarcoid tissues Lancet 350:1655-1661; Ishigi, I. Et al.1999 Quantitative PCR of mycobacterial and propionibacterial DNA in lymph nodes of Japanese patients with sarcoidosis. Lancet 354:120-123. Repeatedly, HLA-class II alleles, especially HLA-DR alleles, have been claimed to be susceptibility genes for sarcoidosis, See Kunikane, H., et al. 1987 Role of HLA-DR antigens in Japanese patients with sarcoidosis Am. Rev. Respir. Dis. 135:688-691; Shousake, A., E. et al. 1987 Association of HLA-DR with sarcoidosis Chest 92: 488-490; Martinetti, M., C. et al. 1995 “The sarcoidosis map”: A joint survey of clinical and imunogenetic findings in two European countries Am. J. Respir Crit Care. Med. 152: 557-564; Berlin, M., A. et al. 1997 HLA-DR predicts the prognosis in Scandinavian patients with sarcoidosis Am. J. Respir Crit. Care Med. 156:1601-1605. These findings and numerous reports of familial case clustering emphasize the important role of genetic background in the development of sarcoidosis. See Headings, V. E., et al. 1976 Familial sarcoidosis with multiple occurrences in eleven families: a possible mechanism of inheritance Ann. N.Y. Acad. Sci. 278: 377-385; Harrington, D. W., et al., 1993 Familial sarcoidosis: analysis of 91 families Sarcoidosis 11:210-243, and differences in inter-ethnic prevalence and phenotypes See Luisetti, M., et al., 2000 Genetic aspects in sarcoidosis. Eur. Respir. J.16: 768-780.

Many advances have recently been made in the understanding of the pathogenesis of sarcoidosis. Accumulation of macrophages and, particularly, CD4-positive T-lymphocytes are present at sites of disease activity, later conglomerating to form granulomata. See Hunninghake, G. W., and R. G. Crystal. 1981 A disorder mediated by excess helper T-lymphocyte activity at sites of disease activity. N. Engl. J. Med. 305:429-434; Hunninghake, G. W., et al. 1981 Characterization of the inflammatory and immune effector cells in the lung parenchyma of patients with interstitial lung disease. Am. Rev. Respir. Dis. 123:407-412. Currently known mechanisms contributing to this cellular expansion include (1) active migration of CD4-positive T-cells and monocytes from blood under the influence of potent chemotactic factors, including MIP-1, MCP-1, RANTES and IL-8. See Standiford, T., et al. 1993 Macrophage inflammatory protein-1α expression in interstitial lung disease. J. Immunol. 151(5): 2852-2863; Car, B. D. et al. 1994 Elevated IL-8 and MCP-1 in the bronchoalveolar lavage fluid of patients with idiopathic pulmonary fibrosis and pulmonary sarcoidosis. Am. J. Respir. Crit Care. Med. 149:655-659; Girgis, R., et al. 1995 Cytokines in the bronchoalveolar lavage fluid of patients with active pulmonary sarcoidosis. Am. J. Respir. Crit. Care Med. 152:71; Lida, K.et al. 1997 Analysis of T cell subsets and β chemokines in patients with pulmonary sarcoidosis Thorax 52:431-437; Kodoma, N., et al.1998 Expression of RANTES by bronchoalveolar lavage cells in nonsmoking individuals with Interstitial lung diseases Am. J. Respir. Cell Mol. Biol. 18:526-531; Ziegenhagen, M. W., et al. 1998 Increased expression of pro-inflammatory cytokines in bronchoalveolar lavage cells of patients with progressing idiopathic pulmonary fibrosis and sarcoidosis, J. Invest. Med. 46:223-231 and (2) in situ-proliferation of lymphocytes (IL-2 mediated). See Hunginghake, G.Wet al. 1983 Role of interleukin-2 release by lung T-cells in active pulmonary sarcoidosis. Am. Rev. Respir. Dis. 128: 634-638; Pinkston, P. et al. 1983 Spontaneous release of interleukin-2 by lung T lymphocytes in active pulmonary sarcoidosis. N. Engl. J. Med. 308:793-800; Semenzato, G., et al.1984 Clin. Exp. Immunol. 57:331-337; Muller-Quernheim, J. et al. 1989. Interleukin-2 receptor gene expression by bronchoalveolar lavage lymphocytes in pulmonary sarcoidosis. Am. Rev. Respir. Dis. 140:82-88. There is evidence of cells (possibly macrophages) bearing intedeukin 2 receptor at sites of disease activity in sarcoidosis. and. See Agostini, C., et al. 1987 Pulmonary alveolar macrophages from patients with sarcoidosis and hypersensitivity pneumonitis: characterization by monoclonal antibodies. J. Clin. Immunol. 7:64-70.

The factors leading to the containment of inflammation in self-limited disease and chronicity and fibrosis in progressive disease remain unexplained. See Cecil Textbook of Medicine, 2^{st} Edition, Goldman L., Bennett, J. C. Editors, W. B. Saunders Company, Philadelphia, 2000 (esp. pages 433-436).

To date, studies in sarcoidosis, have been limited to the examination of a relatively small number of genes, utilizing PCR. However, it is now possible to use high-throughput arrays and confirmatory techniques to shed further light on the critical areas of antigen processing/presentation and recognition, T-lymphocyte and effector cell activation, immunomodulation and tissue remodeling in sarcoidosis. Numerous reports of familial clustering strongly support a genetic susceptibility to sarcoidosis. See Headings, V. E., D. Weston, R. C. Young, and R. L. Hackney. 1976 Familial sarcoidosis with multiple occurrences in eleven families: a possible mechanism of inheritance. Ann. N.Y. Acad. Sci. 278: 377-385; Harrington, D. W., et al. 1993 Familial sarcoidosis: analysis of 91 families. Sarcoidosis 11:210-243; and differences in inter-ethnic prevalence and phenotypes, Luisetti, M., et al. 2000. Genetic aspects in sarcoidosis. Eur. Respir. J. 16: 768-780.

Schurman genotyped 122 affected siblings from 55 German families for 7 DNA polymorphisms that flank and cover the MHC region and found significant multipoint nonparametric linkage for the entire region. See Schurmann, M., et al. 2000 Familial sarcoidosis is linked to the major histocompatibility complex region. Am. J. Respir. Crit. Care Med. 162(3 pt 1):861-864.

The novel discovery, of the present invention, linking the progressive type of sarcoidosis with expression of the HLA-DRB1*1502 gene, adds weight to other studies implicating the MHC complex and, thus, antigen presentation, in susceptibility to, and disease outcome in sarcoidosis. In recent years HLA class 11 molecules have received the most attention in view of their critical role in exogenous antigen presentation and selective activation of CD4-positive T-lymphocytes, a key cell in the pathogenesis of sarcoidosis. In a Japanese study HLA-DR5J (HLA-DRB1*11/12) was significantly associated with non-spontaneous resolution of disease. See Shousake, A., et al. 1987 Association of HLA-DR with sarcoidosis. Chest 92: 488-490; Kunikane, H. et al., 1987 Role of HLA-DR antigens in Japanese patients with sarcoidosis. Am. Rev. Respir. Dis. 135: 688-691.

In an Italian-Czech study HLA-DR4 (HLA-DRB1*04) was significantly associated with type III radiographic disease. See Martinetti, M., et al. 1995 “The sarcoidosis map”: A joint survey of clinical and imunogenetic findings in two European countries. Am. J. Respir Crit Care. Med. 152: 557-564. Interestingly, in a Scandinavian study HLA-DRB1*1501, whilst not significantly over-represented in the sarcoid population as compared to controls, was significantly associated with chronic disease, defined as disease activity 2 years beyond diagnosis (60% vs. 30% in control subjects, p<0.001). See Berlin, M., et al. 1997 HLA-DR predicts the prognosis in Scandinavian patients with sarcoidosis Am. J. Respir Crit. Care Med. 156: 1601-1605. This group has also described a similar association with HLA-DRB1*1401.

The possible mechanisms for this pivotal role of HLA-DR molecules are several. HLA-DRB1*1502 may be an extremely efficient antigen-presenting molecule provoking exaggerated T-cell activation and a self-perpetuating inflammatory cascade. Alternatively, HLA-DRB1*1502 may present antigen inadequately leading to an ineffective primary immune response with resultant antigen intolerance and, therefore, disease chronicity. This latter hypothesis may be the most likely because intolerance of antigen is necessary to maintain the chronic immune response, whereas, highly efficient antigen presentation is more likely to lead to an effective immune response and antigen clearance/tolerance.

The prognostic uncertainty associated with sarcoidosis, has long been a serious problem for clinicians because the only effective treatments known are far from benign. These treatments include high-dose corticosteroids or cytotoxic agents, such as methotrexate, azathioprine, chlorambucil, or cyclophosphamide or other immunosuppressive therapies. These agents have significant toxic effects, including hematologic and gastrointestinal toxicity, teratogenecity and carcinoginicity.

Thus, there is a great need for methods able to discriminate between patients with sarcoidosis who will show spontaneous remission with little or no treatment, and those who will develop the more severe and chronic forms of the disease. In this latter group, aggressive, early treatment is needed to minimize the extent and severity of organ involvement.

### SUMMARY OF THE INVENTION

The present invention overcomes deficiencies in currently available methods to determine the likely severity and chronicity of sarcoidosis by identifying a plurality of genes which are expressed in the peripheral blood mononuclear cells (PBMC's) of patients with the disease. The expression level of these genes can be used to confirm a diagnosis of sarcoidosis or to determine the prognosis, and, therefore, the need for treatment of a patient with sarcoidosis. Thus, the mRNA transcripts and proteins corresponding to these genes have utility, e.g., as surrogate markers or indicators of disease prognosis and need for treatment.

Thus, one aspect of the invention are methods for screening a subject with sarcoidosis to determine the type of disease comprising; obtaining a biological sample from the subject, detecting a level of mRNA expression in said sample corresponding to the gene HLA-DRB1*1502, and determining if the gene HLA-DRB1*1502 is being expressed at a significant level in the said biological sample, wherein the absence of expression at a significant level of said gene identifies Type I sarcoidosis and the presence of expression at a significant level of said gene identifies Type II & III sarcoidosis.

Another aspect of the invention are methods for screening a subject with sarcoidosis to determine the type of disease, comprising; obtaining a biological sample from the subject, detecting a level of expression of the polypeptide product in said sample corresponding to the gene HLA-DRB1*1502, and determining if the polypeptide product in said sample corresponding to the gene HLA-DRB1*1502 is being expressed at a significant level in the said biological sample, wherein the absence of expression at a significant level of said gene identifies Type I sarcoidosis and the presence of expression at a significant level of said gene identifies Type II&III sarcoidosis.

Either of the above methods can employ a biological sample selected from the group consisting of; a tissue biopsy, blood, serum, plasma, lymph, ascitic fluid, cystic fluid, urine, CSF, sputum, or a bronchial aspirate and variety of reagents.

Another aspect of the invention are test kits for use in determining whether sarcoidosis in a patient is Type I or Type II&III, comprising the reagent described above in a container suitable for contacting the biological sample.

In another aspect, the invention provides methods for monitoring the efficacy of a treatment of a subject having sarcoidosis, or at risk of developing sarcoidosis, with an agent, the method comprising; obtaining a pre-administration sample from the subject prior to administration of the agent; detecting a level of expression of mRNA corresponding to a gene selected from the group consisting of those genes identified in Tables 2, 3, 4 or 5, obtaining one or more post-administration samples from the subject; detecting a level of expression of mRNA corresponding to the at least one gene in the post-administration sample or samples; comparing the level of expression of mRNA corresponding to the, at least one, gene in the pre-administration sample with the level of expression of mRNA corresponding to the at last one gene in the post-administration sample; and adjusting the administration of the agent accordingly.

In another aspect, the invention provides methods for monitoring the efficacy of a treatment of a subject having sarcoidosis, or at risk of developing sarcoidosis, with an agent, the method comprising: obtaining a pre-administration sample from the subject prior to administration of the agent; detecting a level of expression of polypeptide encoded by at least one gene selected from the group consisting of those genes identified in Tables 2 or 4, obtaining one or more post-administration samples from the subject; detecting a level of expression of the polypeptide encoded by the at least one gene in the post-administration sample or samples; comparing the level of expression of polypeptide encoded by the at least one gene in the pre-administration sample with the level of expression of polypeptide encoded by the at least one gene in the post-administration sample; and adjusting the administration of the agent accordingly.

In addition, the invention provides methods for identifying agents for use in the treatment of sarcoidosis, comprising; contacting a biological sample obtained from a subject suspected of having sarcoidosis with a candidate agent, detecting a level of expression of a polypeptide encoded by at least one gene in the sample, wherein the gene is selected from the group consisting of those genes identified in Tables 2, 3, 4 or 5, comparing the level of expression of the polypeptide encoded by the at least one gene in the sample in the presence of the candidate agent with a level of expression of the polypeptide encoded by the at least one gene in the sample in the absence of the candidate agent, wherein an altered level of expression of the polypeptide of the at least one gene in the sample in the presence of the candidate agent relative to the level of expression of the polypeptide encoded by the at least one gene in the sample in the absence of the candidate agent is indicative of an agent useful in the treatment of sarcoidosis.

In addition, the invention provides methods for screening a subject for sarcoidosis or at risk of developing sarcoidosis comprising: detecting a level of expression of mRNA corresponding to at least one gene identified in Table 4 in a biological sample obtained from the subject to provide a first value; detecting a level of expression of mRNA corresponding to the at least one gene identified in Table 4 in a similar biological sample obtained from a disease-free subject to provide a second value; and comparing the first value with the second value, wherein a greater first value relative to the second value is indicative of the subject having sarcoidosis or at risk of developing sarcoidosis.

Another aspect of the invention are methods for screening a subject for sarcoidosis or at risk of developing sarcoidosis comprising: detecting a level of expression of a polypeptide, encoded by at least one gene identified in Table 4, in a biological sample obtained from the subject to provide a first value; detecting a level of expression of a polypeptide encoded by the at least one gene identified in Table 4 in a similar biological sample obtained from a disease-free subject to provide a second value; and comparing the first value with the second value, wherein a greater first value relative to the second value is indicative of the subject having sarcoidosis or at risk of developing sarcoidosis.

In addition, the invention provides methods for screening a subject for sarcoidosis or at risk of developing sarcoidosis comprising: detecting a level of expression of mRNA corresponding to at least one gene identified in Table 5, in a biological sample obtained from the subject to provide a first value; detecting a level of expression of mRNA corresponding to the at least one gene identified in Table 5 in a similar biological sample obtained from a disease-free subject to provide a second value; and comparing the first value with the second value, wherein a smaller first value relative to the second value is indicative of the subject having sarcoidosis or at risk of developing sarcoidosis.

Another aspect of the invention provides methods for screening a subject for sarcoidosis or at risk of developing sarcoidosis comprising: detecting a level of expression of a polypeptide encoded by at least one gene identified in Table 5 in a biological sample obtained from the subject to provide a first value; detecting a level of expression of a polypeptide encoded by the at least one gene identified in Table 5 in a similar biological sample obtained from a disease-free subject to provide a second value; and comparing the first value with the second value, wherein a smaller first value relative to the second value is indicative of the subject having sarcoidosis or at risk of developing sarcoidosis.

In addition, the invention provides methods for screening a subject with sarcoidosis to determine the type of disease comprising; detecting a level of expression of mRNA corresponding to at least one gene identified in Table 2, in a biological sample obtained from the subject to provide a first value; detecting a level of expression of mRNA corresponding to the at least one gene identified in Table 2 in a similar biological sample obtained from a disease-free subject to provide a second value; and comparing the first value with the second value, wherein a greater first value relative to the second value is indicative of the subject having Type II & III sarcoidosis.

In a further embodiment, the invention provides methods for screening a subject with sarcoidosis to determine the type of disease comprising: detecting a level of expression of a polypeptide, encoded by at least one gene identified in Table 2, in a biological sample obtained from the subject to provide a first value; detecting a level of expression of a polypeptide, encoded by the at least one gene identified in Table 2, in a similar biological sample obtained from a disease-free subject to provide a second value; and comparing the first value with the second value, wherein a greater first value relative to the second value is indicative of the subject having Type II & III sarcoidosis.

In addition, the invention provides methods for screening a subject with sarcoidosis to determine the type of disease comprising; detecting a level of expression of mRNA corresponding to at least one gene identified in Table 3 in a biological sample obtained from the subject to provide a first value; detecting a level of expression of mRNA corresponding to the at least one gene identified in Table 3 in a similar biological sample obtained from a disease-free subject to provide a second value; and comparing the first value with the second value, wherein a smaller first value relative to the second value is indicative of the subject having Type II & III sarcoidosis.

In addition, the invention provides methods for screening a subject with sarcoidosis to determine the type of disease comprising: detecting a level of expression of a polypeptide encoded by at least one gene identified in Table 3 in a biological sample obtained from the subject to provide a first value; detecting a level of expression of a polypeptide encoded by the at least one gene identified in Table 3 in a similar biological sample obtained from a disease-free subject to provide a second value; and comparing the first value with the second value, wherein a smaller first value relative to the second value is indicative of the subject having Type II & III sarcoidosis.

In a further embodiment the invention provides methods of treating sarcoidosis in a subject in need of such treatment comprising of administering to the subject a compound that modulates the synthesis, expression or activity of one or more of the genes or gene products of the genes shown in Tables 1, 2, 3 or 4 so that at least one symptom of sarcoidosis is ameliorated. The compound may be selected from the group consisting of an antisense molecule, double-stranded RNA, a ribozyme, a small molecule compound, an antibody or a fragment of an antibody.

In addition, the invention provides methods for monitoring the progression of sarcoidosis in a subject having, or at risk of having, sarcoidosis comprising; measuring a level of expression of a polypeptide encoded by at least one gene identified in Tables 2 or 4 over time in a biological sample obtained from the subject, wherein an increase in the level of expression of the polypeptide encoded by the at least one gene over time is indicative of the progression of the sarcoidosis in the subject.

The invention also provides methods for monitoring the progression of sarcoidosis in a subject having, or at risk of having, sarcoidosis, comprising; measuring a level of expression of mRNA corresponding to at least one gene selected from a group consisting of those identified in Tables 2 or 4 over time in a biological sample obtained from the subject, wherein an increase in the level of expression of mRNA of the at least one gene over time is indicative of the progression of the sarcoidosis in the subject.

In a further aspect the invention provides methods for treating patients who have or are suspected of having sarcoidosis comprising: determining the level of expression of mRNA corresponding to the gene expression or the level of polypeptide or protein encoded by one or more of the genes selected from those disclosed in Table 2 or Table 3 in a biological sample from the patient, comparing this level or pattern of levels with levels from normal controls or patients with known types of disease, and treating with immunosuppressive therapy those patients whose level or pattern of levels indicated that they had Type II & III sarcoidosis. This may consist of treatment with cyclosporine at a dose in the range of 1-100 mg/kg/day or 2-50 mg/kg/day or 3-20 mg/kg/day.

### DETAILED DESCRIPTION OF THE INVENTION

The present invention relates to the identification of genes which are differentially expressed in patients with sarcoidosis, as compared with control groups and to the identification of genes which are differentially expressed in patients with Type I sarcoidosis, as compared with patents with Type II & III sarcoidosis.

All specific genes disclosed in the present application particularly those as HLA-DRB1*1502 and the genes of Tables 2, 3, 4, and 5, within the meaning of the invention shall include naturally occurring polymorphic variants thereof. The specifically disclosed genes of Tables 2, 3, 4, and 5 represent preferred embodiments.

As used herein, the term “type of disease” when used in reference to a patient with sarcoidosis shall mean whether the disease is classified as “Type I” or as “Type II & III” as these terms are defined below.

As used herein, “Type I” sarcoidosis refers to patients presenting with symptoms of sarcoidosis whose chest radiographs are staged as 0 or 1 according to the Silzbach classification and, as used herein, “Type II & III” sarcoidosis refers to patients presenting with symptoms of sarcoidosis whose chest radiographs are staged as II, III or IV according to the Silzbach classification. See: Silzbach, L. E. (1967) Med. Clin. N. Am. 51:483; Hunninghake, G. W. (1999) Sarcoidosis, Vasculitis and Diffuse Lung Diseases; 16:149-173 and Cecil Textbook of Medicine, 21^{st} Edition, Goldman L., Bennett, J. C. Editors, W. B. Saunders Company, Philadelphia, 2000 (esp. pages 433-436).

A highly statistically significant correlation has been found between the degree of expression of the genes shown in Tables 2 and 3, i.e., Human MHC class II HLA-DR2-Dw12 mRNA DQw1-beta (HLA-DRB1*1502), Human NF-kappa-B transcription factor p65 subunit (NFKB), Human cyclic AMP-responsive element modulator mRNA (CREM), Human CD69 gene (CD69), Human T-cell receptor alpha chain C region (TRA@) and Human interleukin-10 receptor mRNA-(IL10RA)and the type of disease, i.e., Type I or Type II & III, and therefore the prognosis and need for treatment in patients with sarcoidosis.

Patents with Type II & III sarcoidosis have a much worse prognosis and are much more likely to develop chronic disease than patients with Type I sarcoidosis. In addition, in patients with Type I sarcoldosis the disease is usually self-limited and does not require treatment. In contrast, patients with Type II & III disease are likely to require early intervention with immunosuppressive agents to minimize deleterious long-term effects of the disease.

Therefore, the degree of expression of these genes and the level of their expression products can be used in the management, prognosis and treatment of patients at risk for, or with, sarcoidosis. These genes are identified in Tables 2 and 3. The complete sequences of these genes are available, using the Unigene Cluster expression and GenBank accession numbers shown in Tables 2 and 3.

Detection of abnormally decreased or increased levels of polypeptide or mRNA expression may also be used for diagnosing or determining susceptibility of a subject to a disease of the invention. Methods of detecting the level of expression of mRNA are well known in the art. Decreased or increased expression can be measured at the RNA level using any of the methods well known in the art for the quantitation of polynucleotides, such as, for example, nucleic acid amplification, for instance PCR, RT-PCR, real time quantitative PCR, RNase protection, Northern blotting and other hybridization methods.

A particularly useful method for detecting the level of mRNA transcripts obtained from one or more of the disclosed genes involves hybridization of labeled mRNA to an ordered array of oligonucleotides. Such a method allows the level of transcription of a plurality of these genes to be determined simultaneously to generate gene expression profiles or patterns. The gene expression profile derived from the sample obtained from the subject can, in another embodiment, be compared with the gene expression profile derived form the sample obtained from the disease-free subject, and thereby determine whether the subject has or is at risk of sarcoidosis.

Therefore, this invention provides a plurality of genes that are differentially expressed in patients with different types of sarcoidosis. Any selection, of at least one, of these genes can be utilized as a biomarker to determine the type and prognosis of the disease. In some useful embodiments, a plurality of these genes can be selected and their mRNA expression monitored simultaneously to provide expression profiles for use in various aspects. In a particularly preferred embodiment the level of expression of the gene HLA-DRB1*1502 (Human MHC class II HLA-DR2-Dw12 mRNA DQw1-beta, GenBank accession number M16276) is measured and used as a biomarker to determine the type and prognosis of sarcoidosis. In another preferred embodiment the level of the protein expression product of the gene HLA-DRB1*1502 is measured in a biological sample from a patient with sarcoidosis and this level is used as a biomarker to determine the type and prognosis of the disease.

In addition, statistically significant correlations have been found between the level of gene expression in patients with sarcoidosis, as compared with normal controls. Genes whose expression levels are increased in patients with all types of sarcoidosis, as compared with controls, are shown in Table 4. Genes whose expression levels are decreased in patients with all types of sarcoidosis as compared to controls are shown in Table 5. The complete sequence of these genes is available, using the Unigene Cluster expression and GenBank accession numbers shown in Tables 4 and 5.

Thus, in another embodiment this invention provides a plurality of genes whose expression levels, in patients with sarcoidosis, are significantly higher or lower than in normal individuals. Any selection of at least one of these genes can be utilized as a diagnostic biomarker for sarcoidosis. In addition the difference in expression of these genes in relation to the disease process can be used to identify agents capable of treating the disease and can be used to monitor the extent or progression of the disease. In some useful embodiments, a plurality of these genes can be selected and their mRNA expression (or the polypeptide expression products) monitored simultaneously to provide expression profiles for use in various aspects.

In a further embodiment of the invention, the levels of the gene expression products (proteins or polypeptides) can be monitored in biological samples from patients who have, or are suspected of having, sarcoidosis and from normal controls.

As used herein, the term “biological sample” means any sample of tissue, cells or biological fluid or isolates thereof, which can be removed from the subject for testing, as well as any sample of tissue, cells or biological fluid or isolates thereof within a subject. This includes, but is not limited to, biopsy samples of any kind and cell free lysates of such samples and various body fluids, including, but not limited to; blood, plasma, serum, lymph, CSF, cystic fluid, ascites, urine, stool and bile. Assay techniques that can be used to determine levels of a protein, such as a polypeptide of the present invention, in a sample derived from a host are well-known to those skilled in the art. Such assay methods include radioimmunoassays, competitive-binding assays, Western Blot analysis and ELISA assays.

As used herein, the term “similar biological sample” means a sample from one patient which is from the same general source or location as a sample from another patient. For example, both samples would be of blood, serum, plasma or a biopsy sample from the same organ or body region.

This expression product level can be used to indicate the degree of gene expression and, therefore, can provide a simple and convenient test to determine the diagnosis of sarcoidosis or to determine the type and responsiveness of the subjects disease and the need for immunosuppressive treatment. In addition, the levels of these expression products can be used to identify drugs that may be effective in the treatment of the disease and they may also be used to monitor the severity or progression of the disease in an individual patient.

In addition, expression profiles of one or a plurality of these genes could provide valuable molecular tools for examining the molecular basis for the wide variations in the course and prognosis of sarcoidosis and for evaluating the efficacy of drugs for treating sarcoidosis. Changes in the expression profile from a baseline profile while the cells are exposed to various modifying conditions, such as contact with a drug or other active molecules can be used as an indication of such effects.

Thus, the present invention provides the identification of genes that are differentially expressed in different clinical types of sarcoidosis and in patients with the disease as compared with normal controls. By virtue of the differential expression of these genes, it is possible to utilize these genes and/or their expression products to enhance the certainty of diagnosis, to monitor the severity or progression of the disease or to determine the prognosis of a particular patient and thus whether or not that patient will require immunosuppressive or other therapy and to identify useful new treatments for sarcoidosis. These genes are listed in Tables 2, 3, 4, and 5. The level of expression of the disclosed genes can be detected either by measuring the mRNA corresponding to the gene expression or the polypeptide or protein encoded by the gene. The polypeptide can be measured in any biological sample such as a biopsy specimen or in any convenient body fluid including, but not limited to; blood, plasma, serum, lymph, CSF, cystic fluid, ascites, urine, stool and bile.

Accordingly, this invention provides methods for screening a subject with sarcoidosis to determine the likelihood that the subject's disease will resolve spontaneously or will become chronic and require aggressive treatment, including but not limited to immunosuppressive therapy, such as treatment with high-dose corticosteroids or cytotoxic agents, such as methotrexate, cyclosporine, azathioprine, chlorambucil, or cyclophosphamide.

In addition, this invention provides methods for treating patients who have or are suspected of having sarcoidosis. This method would comprise: 1) determining the level of expression of mRNA corresponding to the gene expression or the level of polypeptide or protein encoded by one or more of the herein disclosed genes in a biological sample from the patient, 2) comparing this level or pattern of levels with levels from normal controls or patients with known types of disease, and 3) treating with immunosuppressive therapy those patients whose level or pattern of levels indicated that they had Type II & III sarcoidosis. In a preferred embodiment this would entail measuring the level of mRNA or the protein expression product of the gene HLA-DRB1*1502 (Human MHC class II HLA-DR2-Dw12 mRNA DQw1-beta, GenBank accession number M16276) in a biological sample from a patient who has or is suspected of having sarcoidosis 2) comparing this level or pattern of levels with levels from normal controls or patients with known types of disease, and 3) treating with immunosuppressive therapy those patients whose level or pattern of levels indicated that they had Type II & III sarcoldosis. This immunosuppressive therapy could include, but would not be limited to, treatment with high-dose corticosteroids or cytotoxic agents, such as cyclosporine, methotrexate, azathioprine, chlorambucil, or cyclophosphamide or other immunosuppressive therapies. In a preferred embodiment the treatment would consist of treatment with cyclosporine at a dose in the range of 1-100 mg/kg/day, or more preferably at a dose in the range or 2-50 mg/kg/day or most preferably at a dose in the range of 3-20 mg/kg/day.

In addition, this invention provides methods for screening a subject to determine if biomarkers for sarcoidosis are present in order to confirm a diagnosis made on purely clinical grounds. This invention also provides methods for monitoring the severity or progression of the disease in an individual and methods to identify new useful treatments for sarcoidosis.

TABLE 2
Genes differentially expressed in Type I versus Type II &
III sarcoidosis (increased in Type II & III)

| | | Unigene | Accession |
|---|---|---|---|
| Gene Symbol | Gene Name | Number | No. |
| 1) HLA-DRB1 * 1502 | Human MHC class II HLA- | 180255 | M16276 |
| | DR2-Dw12 mRNA DQw1- | | |
| | beta | | |
| 2) RELA | Human NF - kappa - B | 75569 | L19067 |
| | transcription factor p65 | | |
| | subunit | | |
| 3) CREM | Human cyclic AMP- | 155924 | S68271 |
| | responsive element | | |
| | modulator mRNA | | |
| 4) CD69 | Human CD69 gene | 82401 | Z22576 |

TABLE 3
Genes differentially expressed in Type I vs. Type II & III
sarcoidosis (decreased in Types II & III)

| Gene | | Unigene | Accession |
|---|---|---|---|
| Symbol | Gene Name | Number | No. |
| 1) TRA@ | Human T-cell receptor alpha chain C | 74647 | X02883 |
| | region | | |
| 2) IL10RA | Human interleukin-10 receptor | 327 | U00672 |
| | mRNA | | |

TABLE 4
Genes expressed at higher levels in patients with all types
of sarcoidosis

| | | Unigene | Accession |
|---|---|---|---|
| Gene Symbol | Gene Name | Number | No. |
| 1) CTSS | Human cathepsin S mRNA | 181301 | M90696 |
| 2) CTSZ | Human cathepsin Z precursor | 252549 | AF032906 |
| | mRNA | | |
| 3) MEP | Human Pro-cathepsin L | 78056 | X12451 |
| 4) IL1B | Human interleukin 1-beta | 126256 | M15330 |
| | mRNA | | |
| 5) IL8 | Human interleukin 8 gene | 624 | M28130 |
| 6) GRO-beta | GRO-beta human cytokine | 75765 | M36820 |
| 7) GRO-gamma | GRO-gamma human cytokine | 89690 | M36821 |
| 8) CCR2 | Chemokine (C—C motif) | 395 | U95626 |
| | receptor 2 | | |
| 9) NINJ1 | Human adhesion molecule | 11342 | U91512 |
| | ninjurin mRNA | | |
| 10) EDN1 | Human endothelin-1 gene | 2271 | J05008 |
| 11) DTR | Human heparin-binding EGF- | 799 | M60278 |
| | like growth factor mRNA | | |
| 12) VEGF | Human vascular endothelial | 73793 | AF024710 |
| | growth factor | | |
| 13) BCL-2 | Human B-cell leukemia | 79241 | M13995 |
| | lymphoma 2 proto-oncogene | | |
| | mRNA | | |
| 14) BCL2A1 | Human Bcl-2 related mRNA | 227817 | U27467 |

TABLE 5
Genes expressed at lower levels in patients with all types
of sarcoidosis

| | | Unigene | Accession |
|---|---|---|---|
| Gene Symbol | Gene Name | Number | No. |
| 1) TNFSFS | Human CD40 ligand gene | 652 | D31797 |
| 2) TNFRSF7 | Human T cell activation antigen | 180841 | M63928 |
| | mRNA | | |
| 3) TOSO | Human anti-Fas-induced apoptosis | 58831 | AF057557 |
| | mRNA | | |
| 4) TNFRSF12 | Human death domain receptor 3 | 180338 | U83598 |
| | soluble form mRNA | | |
| 5) TGFBR2 | Human Tgf-beta IIR alpha | 82028 | D50683 |

### Transcriptional State Measurement

Preferably, measurement of the transcriptional state is made by hybridization to transcript arrays, which are described in this subsection. Certain other methods of transcriptional state measurement are described later in this subsection.

Transcript Arrays Generally

In a preferred embodiment the present invention makes use of “transcript arrays” (also called herein “microarrays”). Transcript arrays can be employed for analyzing the transcriptional state in a cell, and for measuring the transcriptional states of cancer or other disease cells.

In one embodiment, transcript arrays are produced by hybridizing detectably labeled polynucleotides representing the mRNA transcripts present in a cell (e.g., fluorescently labeled cDNA synthesized from total cell mRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics: The arrays are reproducible, allowing multiple copies of a given array to be produced and easily compared with each other. Preferably the microarrays are small, usually smaller than 5 cm.sup.2, and they are made from materials that are stable under binding (e.g. nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. In a specific embodiment, positionally addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

Preparation of Microarrays

Microarrays are known in the art and consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full-length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to testing and confirming a biological network model of interest. A “gene” is identified as an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6275 open reading frames (ORFs) longer than 99 amino acids. Analysis of these ORFs indicates that there are 5885 ORFs that are likely to specify protein products (Goffeau et al., 1996, Life with 6000 genes, Science 274:546-567, which is incorporated by reference in its entirety for all purposes). In contrast, the human genome is estimated to contain approximately 10.sup.5 genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo pl version 5.0 (National Biosciences). In the case of binding sites corresponding to very long genes, it will sometimes be desirable to amplify segments near the 3′ end of the gene so that when oligo-dT primed cDNA probes are hybridized to the microarray; less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well known and are described, for example, in Innis et al. eds., 1990, PCR Protocols: A Guide to Methods and Applications, Academic Press Inc. San Diego, Calif., which is incorporated by reference in its entirety for all purposes. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (Froehler et al., 1986, Nucleic Acid Res 14:5399-5407; McBride et al., 1983, Tetrahedron Lett. 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., 1993, PNA hybridizes to complementary oligonucleotides obeying the Watson-Crick hydrogen-bonding rules, Nature 365:566-568; see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., 1995, Differential gene expression in the murine thymus assayed by quantitative hybridization of arrayed cDNA clones, Genomics 29:207-209). In yet another embodiment, the polynucleotide of the binding sites is RNA.

Attaching Nucleic Acids to the Solid Surface

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., 1995, Quantitative monitoring of gene expression patterns with a complementary DNA microarray, Science 270:467-470. This method is especially useful for preparing microarrays of cDNA. See, also, DeRisi et al., 1996, Use of a cDNA microarray to analyze gene expression patterns in human cancer, Nature Genetics 14:457-460; Shalon et al., 1996, A DNA microarray system for analyzing complex DNA samples using two-color fluorescent probe hybridization, Genome Res. 6:639-645; and Schena et al., 1995, Parallel human genome analysis; microarray-based expression of 1000 genes, Proc. Natl. Acad. Sci. USA 93:10539-11286. Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Light-directed spatially addressable parallel chemical synthesis, Science 251:767-773; Pease et al., 1994, Light-directed oligonucleotide arrays for rapid DNA sequence analysis, Proc. Natl. Acad. Sci. USA 91:5022-5026; Lockhart et al., 1996, Expression monitoring by hybridization to high-density oligonucleotide arrays, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, High-Density Oligonucleotide arrays, Biosensors & Bioelectronics 11: 687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

Generating Labeled Probes

Methods for preparing total and poly(A) .sup.+RNA are well known and are described generally in Sambrook et al., supra. In one embodiment, RNA is extracted from cells of the various types of interest in this invention using guanidinium thiocyanate lysis followed by CsCl centrifugation (Chirgwin et al., 1979, Biochemistry 18:5294-5299). Poly(A) .sup.+RNA is selected by selection with oligo-dT cellulose (see Sambrook et al., supra). Cells of interest include wild-type cells, drug-exposed wild-type cells, cells with modified/perturbed cellular constituent(s), and drug-exposed cells with modified/perturbed cellular constituent(s).

Labeled cDNA is prepared from mRNA by oligo dt-primed or random-primed reverse transcription, both of which are well known in the art (see e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Expression monitoring by hybridization to high-density oligonucleotide arrays, Nature Biotech. 14:1675, which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., 1995, High density cDNA filter analysis: a novel approach for large-scale, quantitative analysis of gene expression, Gene 156:207; Pietu et al., 1996, Novel gene transcripts preferentially expressed in human muscles revealed by quantitative hybridization of a high density cDNA array, Genome Res. 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.™. II, LTI Inc.) at 42.degree. C. for 60 min.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. When the cDNA microarrays of Schena et al. are used, typical hybridization conditions are hybridization in 5×SSC plus 0.2% SDS at 65.degree. C. for 4 hours followed by washes at 25.degree. C. in low stringency wash buffer (1×SSC plus 0.2% SDS) followed by 10 minutes at 25.degree. C. in high stringency wash buffer (0.1×SSC plus 0.2% SDS) (Shena et al., 1996, Proc. Natl. Acad. Sci. USA, 93:10614). Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press San Diego, Calif.

Signal Detection and Data Analysis

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows specimen illumination at wavelengths specific to the fluorophores used and emissions from the fluorophore can be analyzed. In a preferred embodiment, the arrays are scanned with a laser fluorescent scanner with a computer controlled X-Y stage and a microscope objective. Sequential excitation of the fluorophore is achieved with a multi-line, mixed gas laser and the emitted light is split by wavelength and detected with a photomultiplier tube. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site.

If necessary, an experimentally determined correction for “cross talk” (or overlap) between the channels for the two fluors may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

Preferably, in addition to identifying a perturbation as positive or negative, it is advantageous to determine the magnitude of the perturbation. This can be carried out by methods that will be readily apparent to those of skill in the art.

Other Methods of Transcriptional State Measurement

The transcriptional state of a cell may be measured by other gene expression technologies known in the art.

### Taqman™ Based mRNA Levels Analysis

The RT-PCR (real-time quantitative PCR) assay utilizes an RNA reverse transcriptase to catalyze the synthesis of a DNA strand from an RNA strand, including an mRNA strand. The resultant DNA may be specifically detected and quantified and this process may be used to determine the levels of specific species of mRNA. One method for doing this is known under the Trademark TAQMAN (PE Applied Biosystems, Foster City, Calif.) and exploits the 5′ nuclease activity of AMPLI TAQ GOLD™ DNA Polymerase to cleave a specific form of probe during a PCR reaction. This is referred to as a TAQMAN™ probe. See, Luthra R, et al., Novel 5′ exonuclease-based real-time PCR assay for the detection of t(14; 18)(q32;q21) in patients with follicular lymphoma., Am J Pathol., Vol 153, (1998), pp.: 63-68. The probe consists of an oligonucleotide (usually ≅20 mer) with a 5′-reporter dye and a 3′-quencher dye. The fluorescent reporter dye, such as FAM (6-carboxyfluorescein), is covalently linked to the 5′ end of the oligonucleotide. The reporter is quenched by TAMRA (6-carboxy-N,N,N′,N′-tetramethylrhodamine) attached via a linker arm that is located at the 3′ end. See, Kuimelis R G, et al., Structural analogues of TaqMan probes for real-time quantitative PCR., Nucleic Acids Symp Ser., Vol 37, (1997), pp.: 255-256 and Mullah B, et al., Efficient synthesis of double dye-labeled oligodeoxyribonucleotide probes and their application in a real time PCR assay., Nucleic Acids Res., Vol 15, (1998), pp.: 1026-1031. During the reaction, cleavage of the probe separates the reporter dye and the quencher dye, resulting in increased fluorescence of the reporter.

The accumulation of PCR products is detected directly by monitoring the increase in fluorescence of the reporter dye. See Heid C A, et al., Real time quantitative PCR., Genome Res., Vol 6, (1996), pp.: 986-994. Reactions are characterised by the point in time during cycling when amplification of a PCR product is first detected rather than the amount of PCR product accumulated after a fixed number of cycles. The higher the starting copy number of nucleic acid target, the sooner a significant increase in fluorescence is observed. See, Gibson U E, et al., A novel method for real time quantitative RT-PCR., Genome Res., Vol 6, (1996), pp.: 995-1001.

When the probe is intact, the proximity of the reporter dye to the quencher dye results in suppression of the reporter fluorescence primarily by Förster-type energy transfer. See, Lakowicz J R, et al., Oxygen quenching and fluorescence depolarization of tyrosine residues in proteins, J Biol Chem., Vol 258, (1983), pp.: 4794-4801. During PCR, if the target of interest is present, the probe specifically anneals between the forward and reverse primer sites. The 5′-3′ nucleolytic activity of the AMPLITAQ GOLD™ DNA Polymerase cleaves the probe between the reporter and the quencher only if the probe hybridizes to the target. The probe fragments are then displaced from the target, and polymerization of the strand continues. This process occurs in every cycle and does not interfere with the exponential accumulation of product. The 3′ end of the probe is blocked to prevent extension of the probe during PCR.

The passive reference is a dye included in the TAQMAN™ Buffer and does not participate in the 5′ nuclease assay. The passive reference provides an internal reference to which the reporter dye signal can be normalized during data analysis. Normalization is necessary to correct for fluorescent fluctuations due to changes in concentration or volume.

Normalization is accomplished by dividing the emission intensity of the reporter dye by the emission intensity of the passive reference to obtain a ratio defined as the Rₙ (normalized reporter) for a given reaction tube.

The threshold cycle or Cₜ value is the cycle at which a statistically significant increase in ΔRₙ is first detected. On a graph of Rₙ versus cycle number, the threshold cycle occurs when the sequence detection application begins to detect the increase in signal associated with an exponential growth of PCR product.

To perform quantitative measurements serial dilutions of a cRNA (standard) are included in each experiment in order to construct a standard curve necessary for the accurate and fast mRNA quantitation. In order to estimate the reproducibility of the technique the amplification of the same cRNA simple may be performed multiple times.

Other technologies for measuring the transcriptional state of a cell produce pools of restriction fragments of limited complexity for electrophoretic analysis, such as methods combining double restriction enzyme digestion with phasing primers (see, e.g., European Patent 0 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, 1995, Science 270:484-487) pathway pattern.

### Measurement of other Aspects

In various embodiments of the present invention, aspects of the biological state other than the transcriptional state, such as the translational state, the activity state, or mixed aspects can be measured in order to obtain drug and pathway responses. Details of these embodiments are described in this section.

Translational State Measurements

Expression of the protein encoded by the gene(s) can be detected by a probe which is detectably labeled, or which can be subsequently labeled. Generally, the probe is an antibody that recognizes the expressed protein.

As used herein, the term antibody includes, but is not limited to, polyclonal antibodies, monoclonal antibodies, humanized or chimeric antibodies, and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice, and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful-human adjuvants such as BCG (bacille Camette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler and Milstein, Nature, 256:495-497 (1975); and U.S. Pat. No. 4,376,110. The human B-cell hybridoma technique of Kosbor et al., Immunology Today, 4:72 (1983); Cole et al., Proc. Natl. Acad. Sci. USA, 80:2026-2030 (1983); and the EBV-hybridoma technique, Cole et al., Monoclonal Antibodies and Cancer Therapy, Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies”, Morrison et al., Proc. Natl. Acad. Sci USA, 81:6851-6855 (1984); Neuberger et al., Nature, 312:604-608 (1984); Takeda et al., Nature, 314:452-454 (1985), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science, 242:423-426 (1988); Huston et al., Proc. Natl. Acad. Sci. USA, 85:5879-5883 (1988); and Ward et al., Nature, 334:544-546 (1989), can be adapted to produce differentially expressed gene-single chain antibodies. Single chain antibodies are formed by linking the heavy and light chain fragments of the Fv region via an amino acid bridge, resulting in a single chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments, which recognize specific epitopes, may be generated by known techniques. For example, such fragments include, but are not limited to, the F(ab′)₂ fragments which can be produced b pepsin digestion of the antibody molecule and the Fab fragments which can be generated by reducing the disulfide bridges of the F (ab′)₂ fragments. Alternatively, Fab expression libraries may be constructed, Huse et al., Science, 246:1275-1281 (1989), to allow rapid and easy identification of monoclonal Fab fragments with the desired specificity.

The extent to which the known proteins are expressed in the sample is then determined by immunoassay methods that utilize the antibodies described above. Such immunoassay methods include, but are not limited to, dot blotting, western blotting, competitive and noncompetitive protein binding assays, enzyme-linked immunosorbant assays (ELISA), immunohistochemistry, fluorescence activated cell sorting (FACS), and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody must be an antibody that is specific for the protein expressed by the gene of interest.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state may also be performed according to several additional methods. For example, whole genome monitoring of protein (i.e., the “proteome,” Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In a one preferred embodiment, monoclonal antibodies are raised against synthetic peptide fragments designed based on genomic sequence of the cell. With such an antibody array, proteins from the cell are contacted to the array, and their binding is assayed with assays known in the art.

Alternatively, proteins can be separated by two-dimensional gel electrophoresis systems Two-dimensional gel electrophoresis is well known in the art and typically involves Iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Nat'l Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical, or physical means appropriate to the particular activity being characterized. Where the activity involves a chemical transformation, the cellular protein can be contacted with the natural substrates, and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

The Detection of Nucleic Acids and Proteins as Markers

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel, et all, Ed., Current Protocols in Molecular Biology, John Wiley & Sons, New York (1987-1999). Additionally, large numbers of tissue samples can readily be processed using techniques well-known to those of skill in the art, such as, for example, the single-step RNA isolation process of Chomczynski, U.S. Pat. No. 4,843,155 (1989).

The isolated mRNA can be used in hybridization or amplification assays that include, but are not limited to, Southern or Northern analyses, polymerase chain reaction analyses and probe arrays. One preferred diagnostic method for the detection of mRNA levels involve contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7,15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example, by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in Mullis, U.S. Pat. No.4,683,202 (1987); ligase chain reaction, Barany, Proc. Natl. Acad. Sci. USA, 88:189-193 (1991); self-sustained sequence replication, Guatelli et al., Proc. Natl. Acad. Sci. USA, 87:1874-1878 (1990); transcriptional amplification system, Kwoh et al., Proc. Natl. Ac. Sci. USA, 86:1173-1177 (1989); Q-Beta Replicase, Lizardi et al., Bio/Technology, 6:1197 (1988); rolling circle replication, Lizardi et al., U.S. Pat. No. 5,854,033 (1988); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated form the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, or between samples from different sources.

Alternatively, the expression level can be provided as a relative expression level. To determine a relative expression level of a marker, the level of expression of the marker is determined for 10 or more samples of normal versus disease biological samples, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from patients who do not have sarcoidosis. The choice of the cell source is dependent on the use of the relative expression level. Using expression found in normal tissues as a mean expression score aids in validating whether the marker assayed is specific (versus normal cells). In addition, as more data is accumulated, the mean expression value can be revised, providing improved relative expression values based on accumulated data.

Detection of Polypeptides

In another embodiment of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F (ab′) 2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end labeling of a DNA probe with biotin such that it can be detected with fluorescently labeled streptavidin.

Proteins from sarcoidosis patients can be isolated using techniques that are well known to those of skill in the art. The protein isolation methods employed can, for example, be such as those described in Harlow and Lane, Antibodies: A Laboratory Manual, Harlow and Lane, Cold Spring Harbor Laboratory Press, Cold Spring Harbor, N.Y. (1988).

A variety of formats can be employed to determine whether a sample contains a protein that binds to a given antibody. Examples of such formats include, but are not limited to, enzyme immunoassay (EIA); radioimmunoasay (RIA), Western blot analysis and enzyme linked immunoabsorbant assay (ELISA). A skilled artisan can readily adapt known protein/antibody detection methods for use in determining whether cells express a marker of the present invention.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from patient cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker of the invention in a biological sample (e.g., a lung-associated body fluid, serum, plasma, lymph, cystic fluid, urine, stool, csf, acitic fluid, or blood). Such kits can be used to determine prognosis if a subject is suffering from sarcoidosis. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: 1) a first antibody (e.g., attached to a solid support) which binds to a polypeptide corresponding to a marker or the invention; and, optionally, 2) a second, different antibody which binds to either the polypeptide or the first antibody and is conjugated to a detectable label.

For oligonucleotide-based kits, the kit can comprise, for example: 1) an oligonucleotide, e.g., a detectably labeled oligonucleotide, which hybridizes to a nucleic acid sequence encoding a polypeptide corresponding to a marker of the invention; or 2) a pair of primers useful for amplifying a nucleic acid molecule corresponding to a marker of the invention. The kit can also comprise, e.g., a buffering agent, a preservative, or a protein-stabilizing agent. The kit can further comprise components necessary for detecting the detectable label (e.g., an enzyme or a substrate). The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

Introduction of Antibodies into Cells

Antibodies can be introduced into cells in numerous fashions, including, for example, microinjection of antibodies into a cell (Morgan et al., 1988, Immunology Today 9:84-86) or transforming hybridoma mRNA encoding a desired antibody into a cell (Burke et al., 1984, Cell 36:847-858). In a further technique, recombinant antibodies can be engineering and ectopically expressed in a wide variety of non-lymphoid cell types to bind to target proteins as well as to block target protein activities (Biocca et al., 1995, Trends in Cell Biology 5:248-252). Expression of the antibody is preferably under control of a controllable promoter, such as the Tet promoter, or a constitutively active promoter (for production of saturating perturbations). A first step is the selection of a particular monoclonal antibody with appropriate specificity to the target protein (see below). Then sequences encoding the variable regions of the selected antibody can be cloned into various engineered antibody formats, including, for example, whole antibody, Fab fragments, Fv fragments, single chain Fv fragments (V.sub.H and V.sub.L regions united by a peptide linker) (“ScFv” fragments), diabodies (two associated ScFv fragments with different specificity), and so forth (Hayden et al., 1997, Current Opinion in Immunology 9:210-212). Intracellularly expressed antibodies of the various formats can be targeted into cellular compartments (e.g., the cytoplasm, the nucleus, the mitochondria, etc.) by expressing them as fusion's with the various known intracellular leader sequences (Bradbury et al., 1995, Antibody Engineering (vol. 2) (Borrebaeck ed.), pp. 295-361, IRL Press). In particular, the ScFv format appears to be particularly suitable for cytoplasmic targeting.

The Variety of Useful Antibody Types

Antibody types include, but are not limited to, polyclonal, monoclonal, chimeric, single chain, Fab fragments, and an Fab expression library. Various procedures known in the art may be used for the production of polyclonal antibodies to a target protein. For production of the antibody, various host animals can be immunized by injection with the target protein, such host animals include, but are not limited to, rabbit, mice, rats, etc. Various adjuvants can be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacillus Calmette-Guerin (BCG) and corynebacterium parvum.

Monoclonal Antibodies

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein (1975, Nature 256: 495-497), the trioma technique, the human B-cell hybridoma technique (Kozbor et al., 1983, Immunology Today 4: 72), and the EBV hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. USA 80: 2026-2030), or by transforming human B cells with EBV virus in vitro (Cole et al., 1985, in Monoclonal Antibodies and Cancer Therapy, Alan R. Liss, Inc., pp. 77-96). In fact, according to the invention, techniques developed for the production of “chimeric antibodies” (Morrison et al., 1984, Proc. Natl. Acad. Sci. USA 81: 6851-6855; Neuberger et al., 1984, Nature 312:604-608; Takeda et al., 1985, Nature 314: 452-454) by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display (Marks et al., 1992, J. Biol. Chem. 267:16007-16010). Using this technique, libraries of up to 10.sup.12 different antibodies have been expressed on the surface of fd filamentous phage, creating a “single pot” in vitro immune system of antibodies available for the selection of monoclonal antibodies (Griffiths et al., 1994, EMBO J. 13:3245-3260). Selection of antibodies from such libraries can be done by techniques known in the art, including contacting the phage to immobilized target protein, selecting and cloning phage bound to the target, and subcloning the sequences encoding the antibody variable regions into an appropriate vector expressing a desired antibody format.

According to the invention, techniques described for the production of single chain antibodies (U.S. Pat. No. 4,946,778) can be adapted to produce single chain antibodies specific to the target protein. An additional embodiment of the invention utilizes the techniques described for the construction of Fab expression libraries (Huse et al., 1989, Science 246: 1275-1281) to allow rapid and easy identification of monoclonal Fab fragments with the desired specificity for the target protein.

Antibody fragments that contain the idiotypes of the target protein can be generated by techniques known in the art. For example, such fragments include, but are not limited to: the F(ab′).sub.2 fragment which can be produced by pepsin digestion of the antibody molecule; the Fab′ fragments that can be generated by reducing the disulfide bridges of the F (ab′).sub.2 fragment, the Fab fragments that can be generated by treating the antibody molecule with papain and a reducing agent, and Fv fragments.

In the production of antibodies, screening for the desired antibody can be accomplished by techniques known in the art, e.g., ELISA (enzyme-linked immunosorbent assay). To select antibodies specific to a target protein, one may assay generated hybridomas or a phage display antibody library for an antibody that binds to the target protein.

Glossary and Definitions

The following definitions are provided to facilitate understanding of certain terms used frequently hereinbefore.

“Significant level” as used herein, in reference to the level of expression of mRNA or polypeptide product from a particular allele (for example HLA-DRB1*1502) means that level of expression that would lead one of skill in the art to believe that the allele in question was present.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered by the human hands from its natural state, ie. if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons.

“Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds,—i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini.

It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, lie, Leu; Asp, Glu; Asn, Gln-I Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occuring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of which may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity” —For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being—23 compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197,1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length.

Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nim.nih.gov) and FASTA (Pearson W R, Methods in Enzymology, 183, 63-99,1990; Pearson W R and Lipman D J, Proc Nat Acad Sci USA, 85, 2444-2448, 1988, available as part of the Wisconsin Sequence Analysis Package).

Preferably, the BLOSUM62 amino acid substitution matrix (Henikoff S and Henikoff J G, Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutandis for other values of the Identity Index, for instance 0.96, 0.97, 0.98 and 0.99.

The relationship between the number of nucleotide or amino acid differences and the Identity Index may be expressed in the following equation:
nₐ≦xₐ−(xₐ·I)
in which:
nₐ is the number of nucleotide or amino acid differences,
xₐ is the total number of nucleotides or amino acids in SEQ ID NO: 1 or SEQ ID NO: 3 or SEQ ID NO: 2 or SEQ ID NO: 4, respectively,
I is the Identity Index,
· is the symbol for the multiplication operator, and in which any non-integer product of xₐ and
I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotide or polypeptide that within the same species which is functionally similar.

“Fusion protein” refers to a protein encoded by two, unrelated, fused genes or fragments thereof. Examples have been disclosed in U.S. Pat, Nos. 5,541,087 and 5,726,044 (both of which are hereby incorporated by reference for all purposes). In the case of Fc-PGPCR-3, employing an immunoglobulin Fc region as a part of a fusion protein is advantageous for performing the functional expression of Fc-PGPCR-3 or fragments of PGPCR-3, to improve pharmacokinetic properties of such a fusion protein when used for therapy and to generate a dimeric Fc-PGPCR-3. The Fc-PGPCR-3 DNA construct comprises in 5′ to 3′ direction, a secretion cassette, i.e. a signal sequence that triggers export from a mammalian cell, DNA encoding an immunoglobulin Fc region fragment, as a fusion partner, and a DNA encoding Fc-PGPCR-3 or fragments thereof. In some uses it would be desirable to be able to alter the intrinsic functional properties (complement binding, Fc-Receptor binding) by mutating the functional Fc sides while leaving the rest of the fusion protein untouched or delete the Fc part completely after expression.

### EXAMPLE 1

Methods

Twelve consecutive steroid-naive symptomatic patients presenting with acute onset sarcoidosis from August 1999 to August 2000 were included in this prospective study. Patients and 12 healthy controls, matched for age and sex, were non-smokers, non-atopic, with no prior history of malignancy, chronic inflammatory disorder or treatment with corticosteroids. All included subjects were white Caucasians of Celtic origin native to the west of Ireland. Non-atopic state was confirmed by history and negative skin prick challenge to house dust mite, grass pollen, cat and dog dander. After exclusion of inorganic dust exposure, fungal and mycobacterial infection, the diagnosis of sarcoidosis was confirmed histologically in 9 patients by transbronchial lung biopsy and in 3 patients, presenting with erythema nodosum and bilateral hilar lymphadenopathy, by a bronchoalveolar lavage CD4/CD8 ratio >3.5. Costabel, U. 1992. Sensitivity and specificity of BAL findings in sarcoidosis. Sarcoidosis 9(Suppl. 1):211-214; Winterbauer, R.,et al.1993 Bronchoalveolar lavage cell populations in the diagnosis of sarcoidosis. Chest 104:352-361.

All patients had a clinical follow-up at 6 months including chest radiograph and pulmonary function tests. Chest radiographs were staged according to the Silzbach classification. See Silzbach, L. E. 1967 Sarcoidosis: clinical features and management. Med. Clin. N. Am. 51:483. Spirometry and diffusion capacity were measured with a SensorMedics Vmax 22 series.

Peripheral blood mononuclear cells (PBMCs) were separated from 50 mL heparinized whole blood, drawn at the baseline visit, using gradient centrifugation (Ficoll@ Paque, Pharmacia, Uppsala Sweden). The buffy layer was carefully recovered and washed 3 times in culture medium, for example, the commercially available medium sold under the Trademark AIM V©, Life Technologies Paisley, U.K. Cell pellets were frozen and stored at −80° C.

For probe array hybridization experiments the commercially available probe arrays sold under the Trademark GENECHIP® probe arrays (12.6 k Affymetrix), See: Duggan, D. J., et al. 1999 Expression profiling using cDNA microarrays. Nature Genetics 21 (Suppl.1):10-14; DeRisi, J. L., et al. 1996 Use of a cDNA microarray to analyze gene expression patterns in human cancer. Nature Genetics 14:457-460; DeRisi, J. L.,et al. 1997 Exploring the metabolic and genetic control of gene expression on a genomic scale. Science 278:680-686; Brutsche, M. H., et al. 2001 Apoptosis signals in atopy and asthma. Clin Exp Immunol 123(2):181-187; Brutsche, M. H., et al. 2001 B-cell isotype control in atopy and asthma. Am. J. Physiol. Lung Cell Mol Physiol. 280(4):L627-637; were used and extracted RNA from PBMCs was reverse transcribed using, for example, the commercially available kits sold under the Trademark SUPERSCRIPT© Choice System (Life Technologies, USA). The cDNA was then in vitro transcribed, using, for example, the commercially available kits sold under the Trademark (BIOARRAY® HighYield RNA Transcript Labelling Kit, Enzo, USA) to form biotin labelled cRNA. Probe hybridization to the commercially available probe arrays sold under the Trademark GENECHIP® probe arrays (12.6 k Affymetrix), washing, staining and scanning was done according to the instructions of the manufacturer (Affymetrix, USA). For validation of specific genes with altered expression validation experiments were done using real-time RT-PCR, for example, by using the commercially available kits sold under the Trademark (TAQMAN®), TaqMan Reverse Transcription Reagents Kit PE Applied Biosystems (Foster City, Calif.).

HLA haplotype validation experiments at a protein level for class II HLA-DR molecules were performed in a prospective case-controlled association study of 103 patients with biopsy-proven or clinico-radiological features typical of sarcoidosis and 105 healthy controls. Four mL of peripheral blood was drawn into a tube containing 3.2% citrate. Serological typing was performed employing standard lymphocytotoxicity techniques, for example, the commercially available kits sold under the Trademark—HLA Class II Dynabeads® (210.03 Dynal A S, Oslo, Norway), and HLA-DR trays (Gen Trak. Inc. Liberty, N.C., U.S.A.).

For data analysis and mining we used the commercially available data analysis software packages sold under the Trademarks GENE SPRING™ (version 4.0.0, Silicon Genetics) and SPSS® (v10.02, SPSS® Inc). Two-group gene expression comparison between sarcoidosis patients and healthy controls was done using the Mann-Whitney U-test. See Brutsche, M. H., et al. 2001 Apoptosis signals in atopy and asthma. Clin Exp Immunol 123(2):181-187; Brutsche, M. H., et al. 2001 B-cell isotype control in atopy and asthma. Am. J. Physiol. Lung Cell Mol. Physiol. 280(4):L627-637. Only in case of a p-value <0.05 a three-group comparison of healthy controls, Type I and Type II & III sarcoidosis patients (Kruskal-Wallis-test), as well as other two-group comparisons between two phenotypes were performed. Correlation of lung function parameters (DLCO, FEV₁, FVC) and gene expression was analyzed with Spearman' rank correlations. Correlation of results tested by GeneChip®—experiments and TaqMan® were done using Pearson' correlation. A conventional significance level of 0.05 was taken, but required prudent interpretation due to multiple significance testing.

Results

The patients' clinical characteristics are given below in Table 6.

TABLE 6
Patient Characteristics

| | | | | Self-limited | Persistent |
|---|---|---|---|---|---|
| Characteristic | | Controls | Patients | sarcoidosis | sarcoidosis |
| [n] | | 12 | 12 | 7 | 5 |
| Age [years] | | 32 ± 8 | 36 ± 12 | 33 ± 11 | 39 ± 13 |
| Sex [m:f] | | 5:7 | 5:7 | 4:3 | 1:4 |
| Mean duration of | | | 3.7 ± 4.9 | 1.3 ± 0.8 | 7.9 ± 7.0 |
| symptoms pre | | | [0.75-18] | [0.8-2.5] | [2.5-18] |
| diagnosis | | | | | |
| [months ± SD | | | | | |
| (range)] | | | | | |
| Mean follow-up | | | 14.3 ± 4.4 | 13.6 ± 4.5 | 15.3 ± 4.6 |
| [months ± SD | | | [7.5-12] | [7.5-20] | [2.5-18] |
| (range)] | | | | | |
| Erythema | | | | 7 | 0 |
| nodosum | | | | | |
| Silzbach score at | | | | | |
| baseline [n] | | | | | |
| 1 | | | | 7 | |
| 2 | | | | 0 | 3 |
| 3 | | | | 0 | 2 |
| Immuno- | | | | 0 (0) | 5 (100) |
| suppressive | | | | | |
| treatment [n (%)] | | | | | |
| Pulmonary | | | | | |
| Function | | | | | |
| [% pred. ± SD] | | | | | |
| FVC, % | baseline | | | 102 ± 11 | 84 ± 26 |
| | Follow- | | 107 ± 9 | 93 ± 11 | |
| | up | | | | |
| FEV, % | baseline | | | 97 ± 19 | 89 ± 25 |
| | Follow- | | 101 ± 10 | 88 ± 16 | |
| | up | | | | |
| TLCO, | baseline | | | 97 ± 13 | 66 ± 26 |
| % | Follow- | | 101 ± 7 | 68 ± 16 | |
| | up | | | | |

Antigen processing for HLA class II molecules—We found a consistent up-regulation of cathepsin genes L, S and Z in patients as compared to controls.

Antigen presentation—8/12 (67%) healthy control subjects were HLA-DR15*1502-positive. None of the patients with self-limited disease, but all patients with persistent disease were HLA-DRB1*1502-positive. There was significant down-regulation of expression of the T-cell receptor alpha chain C region in patients with Type II&III disease compared to controls.

Co-stimulation—Expression of co-stimulatory gene CD40L was significantly down-regulated in patients compared to controls. There was no difference in the expression of the co-stimulatory molecules CD28, CD80 and CD86.

T-cell activation—CD71 (transferrin receptor) was up-regulated in all patients compatible with recent T-cell activation. Stronger T-cell activation was found in patients with persistent disease and CD69 was up-regulated in these patients. The CD27 gene was down-regulated in all patients compared to controls. There was no difference for expression of IFN-gamma, IL2 and IL2RB chain receptor genes.

Transcription factors—Expression of NFKB and CREM were significantly up-regulated in patients with Type II&III disease.

Effector cell activation—Expression of IL1 and IL8 genes, which are mainly expressed and secreted by monocytes and macrophages, was consistently up-regulated in sarcoidosis patients. Similarly, TNF-alpha expression was up-regulated in patients with Type II and III sarcoidosis. Expression of B-cell derived immunoglobulin genes showed upregulated immunoglobulin-G and Fc-gamma-receptor and down-regulated immunoglobulin-E and high-affinity Fc-epsilon RI receptor, indicative of a IgG1 B-cell isotype predominance.

Cell migration and adhesion—The chemokine receptor gene complex CCR2/CCR5/CCR6 and GRO beta and gamma genes were significantly up-regulated, particularly in patients with Type I disease. In Type II&III disease due to higher expression variability the up-regulation for GRO beta and gamma genes did not reach statistical significance. Expression of chemokine MCP was not altered compared to controls. Of the adhesion molecules measured, expression of the ninjurin, integrin beta-5 and alpha-3, as well as CD11b (CCR3) was up-regulated.

| | Sarcoidosis | | HLA-DR2- | |
|---|---|---|---|---|
| Patient number | type | beta actin | Dw12 | Ratio * 10⁵ |
| 19 | Type I | 3.9E+08 | ND | |
| 23 | Type I | 6.1E+07 | ND | |
| 33 | Type I | 8.7E+08 | ND | |
| 34 | Type I | 6.1E+08 | ND | |
| 35 | Type I | 4.8E+08 | ND | |
| 36 | Type I | 1.0E+09 | ND | |
| 39 | Type I | 2.7E+08 | ND | |
| 22 | Type II & III | 9.3E+07 | 2.2E+04 | 23.66 |
| 24 | Type II & III | 2.0E+08 | 7.5E+03 | 3.75 |
| 25 | Type II & III | 2.3E+08 | 8.1E+03 | 3.52 |
| 38 | Type II & III | 5.6E+08 | 3.8E+04 | 6.79 |

Discussion

With this high-density gene array approach in peripheral blood, it was possible to demonstrate clear differences in gene expression between healthy individuals and patients with acute onset pulmonary sarcoidosis. The findings indicate increased antigen-processing/presentation, activation of T-lymphocytes, activation of effector cells (B-lymphocytes, monocytes), increased cell migration & tissue remodeling, together with altered inflammatory containing mechanisms.

Furthermore, the results demonstrated specific differences between sarcoidosis patients with progressive disease compared to those with a self-limited form of disease. In particular, HLA-DRB1*1502 positivity was associated with chronic and more severe disease, the need for immunosuppressive therapy and a worse prognosis. Conversely, all patients not expressing HLA-DRB1*1502 had complete spontaneous disease regression within 6-12 months, suggesting that differences in antigen-presentation may alter the immune response, either provoking an acutely flaring, spontaneously subsiding response in the absence of HLA-DRB1*1502 or a more low-grade chronic form of immune response in its presence. In a prospective association study in a cohort of 103 sarcoidosis patients the importance of HLA DR2 (HLA DRB1*15/16) both in disease susceptibility and as a predictor of poor outcome was confirmed.

Underlining the existence of a differential activation of the immune system In patients with self-limited sarcoidosis are the more markedly upregulated IL1B and chemokines/receptors (GRO-beta/-gamma, CCR2/5/6) In Type I disease, as well as the more pronounced markers of T-lymphocyte activation and upregulated transcription factors NFKB and CREM in patients with Type II & III-sarcoidosis. Knock-out mice models have shown the importance of NFKB for Th1-specific delayed-type hypersensitivity reactions, See Aronica, M. A., et al. 1999 Preferential role for NF-kappa B/Rel signalling In the type 1 but not type 2 T cell-dependant immune response in vivo. J. Immunol. 63:5116-5124; as observed in sarcoldosis. These findings are in accordance with an immune response for the elimination of antigen, which for Type I disease is more effector cell-based, whereas Type II & III disease depends more on T-helper lymphocyte activation with a more pronounced, chronic delayed-type hypersensitivity reaction.

References Cited

All publications and references, including but not limited to publications, patents, patent applications, GenBank accession, Unigene Cluster numbers and protein accession numbers, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method for screening a subject with sarcoidosis to determine the type of disease comprising;
(a) obtaining a biological sample from the subject, (b) detecting a level of mRNA expression in said sample corresponding to the gene HLA-DRB1*1502,
(c) determining if the gene HLA-DRB1*1502 is being expressed at a significant level in the said biological sample, wherein the absence of expression at a significant level of said gene identifies Type I sarcoidosis and the presence of expression at a significant level of said gene identifies Type II&III sarcoidosis.

2. A method for screening a subject with sarcoidosis to determine the type of disease comprising;
(a) obtaining a biological sample from the subject,
(b) detecting a level of expression of the polypeptide product in said sample corresponding to the gene HLA-DRB1*1502,
(c) determining if the polypeptide product in said sample corresponding to the gene HLA-DRB1*1502 is being expressed at a significant level in the said biological sample, wherein the absence of expression at a significant level of said gene identifies Type I sarcoidosis and the presence of expression at a significant level of said gene identifies Type II&III sarcoidosis.

3. The method of claim 1, wherein the biological sample is selected from the group consisting of; a tissue biopsy, blood, serum, plasma, lymph, ascitic fluid, cystic fluid, urine, CSF, sputum, or a bronchial aspirate.

4. A method according to claim 3 wherein said biological sample is a tissue biopsy cell sample or cells cultured therefrom.

5. A method according to claim 4, wherein said sample is a lysate of said cell sample.

6. The method of claim 2, wherein the presence of the polypeptide is detected using a reagent which specifically binds with the polypeptide.

7. The method of claim 6, wherein the reagent is selected from the group consisting of an antibody, an antibody derivative, and an antibody fragment.

8. A test kit for use in determining whether sarcoidosis in a patient is Type I or Type II&III, comprising the reagent of claim 6 in a container suitable for contacting the biological sample.

9. The test kit of claim 8, wherein the reagent comprises an antibody, in which case the antibody specifically binds with a polypeptide corresponding to the gene expression product.

10. The method of claim 2, wherein the level of expression of the polypeptide encoded by the gene HLA-DRB1*1502 is detected through Western blotting by utilizing a labeled probe specific for the polypeptide.

11. The method of claim 10, wherein the labeled probe is an antibody.

12. The method of claim 11, wherein the antibody is a monoclonal antibody.

13. The method of claim 1, wherein the level of expression of mRNA is detected by techniques selected from the group consisting of Northern blot analysis, reverse transcription PCR, real time quantitative PCR, RNase protection and microarray.

14. A method for monitoring the efficacy of a treatment of a subject having sarcoidosis, or at risk of developing sarcoidosis, with an agent, the method comprising;
a) obtaining a pre-administration sample from the subject prior to administration of the agent;
b) detecting a level of expression of mRNA corresponding to a gene selected from the group consisting of those genes identified in Tables 2, 3, 4 or 5.
c) obtaining one or more post-administration samples from the subject;
d) detecting a level of expression of mRNA corresponding to the at least one gene in the post-administration sample or samples;
e) comparing the level of expression of mRNA corresponding to the at least one gene in the pre-administration sample with the level of expression of mRNA corresponding to the at last one gene in the post-administration sample; and
f) adjusting the administration of the agent accordingly.

15. A method for monitoring the efficacy of a treatment of a subject having sarcoidosis, or at risk of developing sarcoidosis, with an agent, the method comprising:
a) obtaining a pre-administration sample from the subject prior to administration of the agent;
b) detecting a level of expression of polypeptide encoded by at least one gene selected from the group consisting of those genes identified in Tables 2 or 4
c) obtaining one or more post-administration samples from the subject;
d) detecting a level of expression of the polypeptide encoded by the at least one gene in the post-administration sample or samples;
e) comparing the level of expression of polypeptide encoded by the at least one gene in the pre-administration sample with the level of expression of polypeptide encoded by the at least one gene in the post-administration sample; and
f) adjusting the administration of the agent accordingly.

16. A method for identifying agents for use in the treatment of sarcoidosis, comprising;
a) contacting a biological sample obtained from a subject suspected of having sarcoidosis with a candidate agent,
b) detecting a level of expression of a polypeptide encoded by at least one gene in the sample, wherein the gene is selected from the group consisting of those genes identified in Tables 2, 3, 4 or 5,
c) comparing the level of expression of the polypeptide encoded by the at least one gene in the sample in the presence of the candidate agent with a level of expression of the polypeptide encoded by the at least one gene in the sample in the absence of the candidate agent, wherein an altered level of expression of the polypeptide of the at least one gene in the sample in the presence of the candidate agent relative to the level of expression of the polypeptide encoded by the at least one gene in the sample in the absence of the candidate agent is indicative of an agent useful in the treatment of sarcoidosis.

17. The method of claim 16, wherein the level of expression of the polypeptide encoded by the at least one gene is detected through Western blotting by utilizing a labeled probe specific for the polypeptide.

18. The method of claim 17, wherein the labeled probe is an antibody.

19. The method of claim 18, wherein the antibody is a monoclonal antibody.

20. The method of claim 16, wherein the agent is selected from the group consisting of small molecules.

21. A method for screening a subject for sarcoidosis or at risk of developing sarcoidosis comprising:
a) detecting a level of expression of mRNA corresponding to at least one gene identified in Table 4 in a biological sample obtained from the subject to provide a first value;
b) detecting a level of expression of mRNA corresponding to the at least one gene identified in Table 4 in a similar biological sample obtained from a disease-free subject to provide a second value; and
c) comparing the first value with the second value, wherein a greater first value relative to the second value is indicative of the subject having sarcoidosis or at risk of developing sarcoidosis.

22. The method of claim 21 wherein the biological sample is selected from the group consisting of; a tissue biopsy, blood, serum, plasma, lymph, ascitic fluid, cystic fluid, urine, CSF, sputum, or a bronchial aspirate.

23. The method of claim 21, wherein the level of expression of mRNA in steps (a) and (b) is detected by techniques selected from the group consisting of Northern blot analysis, reverse transcription PCR, real time quantitative PCR, RNase protection and microarray.

24. A method for screening a subject for sarcoidosis or at risk of developing sarcoidosis comprising:
a) detecting a level of expression of a polypeptide encoded by at least one gene identified in Table 4 in a biological sample obtained from the subject to provide a first value;
b) detecting a level of expression of a polypeptide encoded by the at least one gene identified in Table 4 in a similar biological sample obtained from a disease-free subject to provide a second value; and
c) comparing the first value with the second value, wherein a greater first value relative to the second value is indicative of the subject having sarcoidosis or at risk of developing sarcoidosis.

25. The method of claim 24 wherein the biological sample is selected from the group consisting of; a tissue biopsy, blood, serum, plasma, lymph, ascitic fluid, cystic fluid, urine, CSF, sputum, or a bronchial aspirate.

26. The method of claim 24 wherein the level of expression of the polypeptide in steps (a) and (b) is detected through Western blotting by utilizing a labeled probe specific for the polypeptide.

27. The method of claim 24, wherein the probe is an antibody.

28. The method of claim 27, wherein the antibody is a monoclonal antibody.

29. A method for screening a subject for sarcoidosis or at risk of developing sarcoidosis comprising:
a) detecting a level of expression of mRNA corresponding to at least one gene identified in Table 5 in a biological sample obtained from the subject to provide a first value;
b) detecting a level of expression of mRNA corresponding to the at least one gene identified in Table 5 in a similar biological sample obtained from a disease-free subject to provide a second value; and
c) comparing the first value with the second value, wherein a smaller first value relative to the second value is indicative of the subject having sarcoidosis or at risk of developing sarcoidosis.

30. The method of claim 29 wherein the level of expression of mRNA in steps (a) and (b) is detected by techniques selected from the group consisting of Northern blot analysis, reverse transcription PCR and real time quantitative PCR.

31. A method for screening a subject for sarcoidosis or at risk of developing sarcoidosis comprising:
a) detecting a level of expression of a polypeptide encoded by at least one gene identified in Table 5 in a biological sample obtained from the subject to provide a first value;
b) detecting a level of expression of a polypeptide encoded by the at least one gene identified in Table 5 in a similar biological sample obtained from a disease-free subject to provide a second value; and
c) comparing the first value with the second value, wherein a smaller first value relative to the second value is indicative of the subject having sarcoidosis or at risk of developing sarcoidosis.

32. The method of claim 31, wherein the level of expression of the polypeptide in steps (a) and (b) is detected through Western blotting by utilizing a labeled probe specific for the polypeptide.

33. The method of claim 32, wherein the probe is an antibody.

34. The method of claim 33, wherein the antibody is a monoclonal antibody.

35. A method for screening a subject with sarcoidosis to determine the type of disease comprising;
a) detecting a level of expression of mRNA corresponding to at least one gene identified in Table 2 in a biological sample obtained from the subject to provide a first value;
b) detecting a level of expression of mRNA corresponding to the at least one gene identified in Table 2 in a similar biological sample obtained from a disease-free subject to provide a second value; and
c) comparing the first value with the second value, wherein a greater first value relative to the second value is indicative of the subject having Type II & III sarcoidosis.

36. The method of claim 35, wherein the level of expression of mRNA in steps (a) and (b) is detected by techniques selected from the group consisting of Northern blot analysis, reverse transcription PCR and real time quantitative PCR.

37. A method for screening a subject with sarcoidosis to determine the type of disease comprising:
a) detecting a level of expression of a polypeptide encoded by at least one gene identified in Table 2 in a biological sample obtained from the subject to provide a first value;
b) detecting a level of expression of a polypeptide encoded by the at least one gene identified in Table 2 in a similar biological sample obtained from a disease-free subject to provide a second value; and
c) comparing the first value with the second value, wherein a greater first value relative to the second value is indicative of the subject having Type II & III sarcoidosis.

38. The method of claim 37, wherein the level of expression of the polypeptide in steps (a) and (b) is detected through Western blotting by utilizing a labeled probe specific for the polypeptide.

39. The method of claim 38, wherein the probe is an antibody.

40. The method of claim 39, wherein the antibody is a monoclonal antibody.

41. A method for screening a subject with sarcoidosis to determine the type of disease comprising;
a) detecting a level of expression of mRNA corresponding to at least one gene identified in Table 3 in a biological sample obtained from the subject to provide a first value;
b) detecting a level of expression of mRNA corresponding to the at least one gene identified in Table 3 in a similar biological sample obtained from a disease-free subject to provide a second value; and
c) comparing the first value with the second value, wherein a smaller first value relative to the second value is indicative of the subject having Type II & III sarcoidosis.

42. The method of claim 41 wherein the level of expression of mRNA in steps (a) and (b) is detected by techniques selected from the group consisting of Northern blot analysis, reverse transcription PCR and real time quantitative PCR.

43. A method for screening a subject with sarcoidosis to determine the type of disease comprising:
a) detecting a level of expression of a polypeptide encoded by at least one gene identified in Table 3 in a biological sample obtained from the subject to provide a first value;
b) detecting a level of expression of a polypeptide encoded by the at least one gene identified in Table 3 in a similar biological sample obtained from a disease-free subject to provide a second value; and
c) comparing the first value with the second value, wherein a smaller first value relative to the second value is indicative of the subject having Type II & III sarcoidosis.

44. The method of claim 43 wherein the level of expression of the polypeptide in steps (a) and (b) is detected through Western blotting by utilizing a labeled probe specific for the polypeptide.

45. The method of claim 44, wherein the probe is an antibody.

46. The method of claim 45, wherein the antibody is a monoclonal antibody.

47. A method of treating sarcoidosis in a subject in need of such treatment comprising of administering to the subject a compound that modulates the synthesis, expression or activity of one or more of the genes or gene products of the genes shown in Tables 1, 2, 3 or 4 so that at least one symptom of sarcoidosis is ameliorated.

48. The method of claim 20, wherein the compound is selected from the group consisting of an antisense molecule, double-stranded RNA, a ribozyme, a small molecule compound, an antibody or a fragment of an antibody.

49. A method for monitoring the progression of sarcoidosis in a subject having, or at risk of having, sarcoidosis comprising; measuring a level of expression of a polypeptide encoded by at least one gene identified in Tables 2 or 4 over time in a biological sample obtained from the subject, wherein an increase in the level of expression of the polypeptide encoded by the at least one gene over time is indicative of the progression of the sarcoidosis in the subject.

50. A method for monitoring the progression of sarcoidosis in a subject having, or at risk of having, sarcoidosis, comprising; measuring a level of expression of mRNA corresponding to at least one gene selected from a group consisting of those identified in Tables 2 or 4 over time in a biological sample obtained from the subject, wherein an increase in the level of expression of mRNA of the at least one gene over time is indicative of the progression of the sarcoidosis in the subject.

51. The method of claim 50, wherein the level of expression of mRNA is detected by techniques selected from the group consisting of Northern blot analysis, reverse transcription PCR,real time quantitative PCR, RNAse protection assay, and microarray.

52. The method of claim 50, wherein the at least one gene is selected from the group consisting of; Human NF-kappa-B transcription factor p65 subunit (NFKB), Human cyclic AMP-responsive element modulator mRNA (CREM), Human cyclic AMP-responsive element modulator beta isoform S68134 (CREM, beta isoform), Human CD69 gene (CD69).

53. The method of claim 50, wherein the at least one gene is selected from the group consisting of; Human T-cell receptor alpha chain C region and Human interleukin-10 receptor mRNA.

54. A method for treating patients who have or are suspected of having sarcoidosis comprising: 1) determining the level of expression of mRNA corresponding to the gene expression or the level of polypeptide or protein encoded by one or more of the genes selected from those disclosed in Table 2 or Table 3 in a biological sample from the patient, 2) comparing this level or pattern of levels with levels from normal controls or patients with known types of disease, and 3) treating with immunosuppressive therapy those patients whose level or pattern of levels indicated that they had Type II&III sarcoidosis.

55. The method according to claim 54 wherein the said gene is HLA-DRB1*1502 (Human MHC class II HLA-DR2-Dw12 mRNA DQw1-beta, GenBank accession number M16276).

56. The method according to claim 55 wherein the said immunosuppresive therapy would be selected from a group consisting of; corticosteroids, cyclosporine, methotrexate, azathioprine, chlorambucil, or cyclophosphamide.

57. The method according to claim 56 wherein the said immunosuppresive therapy consists of treatment with cyclosporine at a dose in the range of 1-100 mg/kg/day.

58. The method according to claim 56 wherein the said immunosuppresive therapy consists of treatment with cyclosporine at a dose in the range or 2-50 mg/kg/day.

59. The method according to claim 56 wherein the said immunosuppresive therapy consists of treatment with cyclosporine at a dose of 3-20 mg/kg/day.

60. A kit for the identification of sarcoidosis patients comprising means for detecting the level of expression of mRNA corresponding to the gene HLA-DRB1*1502.

61. A kit for the identification of sarcoidosis patients comprising means for detecting the level of expression of mRNA corresponding to at least one gene identified in Tables 2, 3, 4 or 5.

62. A kit for identifying whether a sarcoidosis patient is Type I or Type II&III, comprising means for detecting the level of expression of mRNA corresponding to at least one gene identified in Tables 2 or 3.

63. A kit according to claim 60, wherein the level of expression of mRNA is detected by techniques selected from the group of Northern blot analysis, reverse transcriptase PCR, real-time PCR, RNAase protection, and microarray.

64. A kit for the identification of sarcoidosis patients comprising means for detecting the level of expression of the polypeptide encoded by the gene HLA-DRB1*1502.

65. A kit for the identification of sarcoidosis patients comprising means for detecting a level of expression of a polypeptide encoded by at least one gene identified in Tables 2, 3, 4 or 5.

66. A kit for identifying whether a sarcoidosis patient is Type I or Type II&III, comprising means for detecting a level of expression of a polypeptide encoded by at least one gene identified in Tables 2 or 3.

67. A kit according to claim 64, wherein the level of expression of said polypeptide is detected through Western blotting utilizing a labeled probe specific for said polypeptide.

68. A kit according to claim 67, wherein the labeled probe is an antibody.

69. A kit according to claim 68, wherein the antibody is a monoclonal antibody.

70. A kit according to claims 60, further comprising means for obtaining a biological sample from a subject.
